(19) United States
(12) Patent Application Publication
(10) Pub. No.: US 2022/0373529 A1
(43) Pub. Date: Nov. 24, 2022

(54) SENSOR AND METHOD FOR DETECTING HEAVY METALS USING CARBON NANOTUBES

(71) Applicant: Morgan State University, Baltimore, MD (US)

(72) Inventor: Dereje Seifu, Towson, MD (US)

(21) Appl. No.: 17/732,975

(22) Filed: Apr. 29, 2022

Related U.S. Application Data
(60) Provisional application No. 63/181,469, filed on Apr. 29, 2021.

(51) Int. Cl. G01N 33/18; G01N 23/085; G01N 23/202; C02F 1/62; C02F 1/28

(52) U.S. Cl. CPC G01N 33/1813; G01N 23/085; G01N 23/202; C02F 1/62; C02F 1/288; C02F 2101/22

(57) ABSTRACT

Sensor and method for detecting, monitoring and/or removing trace amounts of heavy metal in a liquid. The sensor including magnetic nanoparticle composites of carbon nanotubes intercalated with CoFe₂O₄; the method including contacting a sample of liquid with the magnetic nanoparticle composites and measuring the X-ray diffraction and magnetic properties of the magnetic nanoparticle composite, where a statistical difference in the X-ray diffraction or magnetic properties of the magnetic nanoparticle composite before and after contact between said sample of said liquid and said magnetic nanoparticle composite indicates the presence of a heavy metal in said liquid.

### STATEMENT OF GOVERNMENT RIGHTS

This invention was made with Government support under contract number USARMY-W911NF-12-02-0041 awarded by US Army Research Laboratory. The Government has certain rights in this invention.

### BACKGROUND OF THE INVENTION

### Field of the Invention

The present invention relates to sensors and methods for measuring, monitoring and removing heavy metals in/from liquids, especially water.

### Description of the Background

U.S. Pat. No. 7,342,479 describes chemically sensitive resistors that utilize carbon nanotubes for detecting analytes in a fluid. U.S. Pat. No. 8,920,619 relates to carbon nanotube sensors but primarily describes a remote monitoring system for monitoring the operation of a fluid treatment system.

Publications include “Detection of Trace Heavy Metal Ions Using Carbon Nanotube-Modified Electrodes,” by Morton, et al, which detected lead and copper in spiked lake water; and “Highly sensitive detection of hexavalent chromium utilizing a sol-gel/carbon nanotube modified electrode,” by Rosolina, et al., is a new development in the electroanalytical field.

### SUMMARY OF THE INVENTION

The invention is a sensor and method for the detection, monitoring and/or removal of heavy metals in liquids such as water, the sensor composed of carbon nanotubes in which magnetic nanoparticles of cobalt ferrite have been embedded in layers of the nanotube structure.

Heavy metals in tiny amounts can be detected from a contaminated water source. More specifically, carbon nanotubes (CNTs) intercalated with magnetic nanoparticles of cobalt ferrite CoFe₂O₄, or CFOs, are used for the detection, identification, and quantitation of chromium by observing changes in the magnetic properties of the composite.

Heavy metals include chromium, arsenic, cadmium, mercury, and lead, all of which are found in drinking water. Absorption of these elements and related compounds into the human body poses serious risks of contracting cancer; thus, it is important to have a sensor that has the ability to detect and quantify whether the amounts in water are within federal standards.

Chromium is generally found as one of over twenty types of isotopes, and of those, three isotopes are stable. Chromium is also found in ionic form, and the most common are chromium trivalent (Cr-III) and chromium hexavalent (Cr-VI). Cr-VI is considered a significant toxin, with exposure to this compound via inhalation, ingestion, or contact with the skin as possible paths towards renal damage, allergies, asthma, gastrointestinal hematological, neurological damage, and cancer of the respiratory tract.

A Vibrating Sample Magnetometer (VSM) was used to measure the magnetization of the composite under an applied magnetic field (H) between −15000 and 15000 Oersted (Oe). The same sample was then treated with deionized (DI) water to investigate the change in magnetization. The sample was then infiltrated with K₂Cr₂O₇ to represent chromium hexavalent under the exact condition of VSM. The data were compared for the differences in magnetization.

For VSM characterization, the CNTs and CFO were intercalated by ultrasonication. The CNT-CFO composite sample was prepared for the study of the magnetic characteristics. First, the VSM powder cup containing CNT-CFO was a load to the VSM, and data were collected for magnetic measurements. The VSM powder cup containing CNT-CFO was treated with 70 μl of DI water and left overnight for air dry. The magnetic properties of the sample were measured using VSM. The sample was then infiltrated with K₂Cr₂O₇. A 0.2 g of K₂Cr₂O₇ was dispersed in 65 ml of deionized water, equivalent to 71 mg of chromium. The K₂Cr₂O₇ solution was mixed using a magnetic stirrer for 1 hour, and 90 μl of the mixture was transferred to the CNT-CFO powder cup. A 90 μl of the mixture contains 98 μl of chromium. CNT-CFO-K₂Cr₂O₇ sample was dried overnight for solvent evaporation. Then, the VSM powder cup containing the sample was loaded on the VSM for measuring magnetic properties.

The magnetic properties of CNT-CFO without chromium and CNT-CFO infiltrated with K₂Cr₂O₇ were studied using VSM at room temperature and compared to each other. Three VSM measurements were carried out, and the first was for pristine CNT-CFO; pristine CNT-CFO refers to a composite made from carbon nanotubes and cobalt ferrite intercalated with ultrasonication. On the second VSM measurement, the same sample was treated by adding 90 μl of DI water and dried at room temperature. The third sample was prepared by infiltrating the second sample with 90 μl of K₂Cr₂O₇. By calculation, 90 μl of the mixture contains about 98 μg of chromium. The sample was then dried in the hood at room temperature. The magnetic properties were measured at a maximum applied field of 15 kOe. The change in the composite CNT-CFO's magnetic properties can be used as one of the most critical methods of studying substances and their function.

The VSM powder cup contains a CNT-CFO nanoparticle with a mass of 0.0312 grams. The powder cup is cylindrical, 6.0 mm in diameter, and 3.10 mm in length. The magnetization was divided by the mass of CNT-CFO to calculate the average magnetization in electromagnetic unit per gram (emu/g) of the sample. The actual mass of chromium is 98 μg. The composite sample's magnetization per gram (CNT-CFO-K₂Cr₂O₇) in emu divided by the total mass of CNT-CFO and chromium, which is (31.2 mg+0.098 mg) equals 31.3 mg. The average parameters are averaged by 31.2 mg of CNT-CFO, 98 μg of chromium, and 3.75 g of surfactant (Sodium Dodecylbenzene Sulfonate, or SDBS). The CNTs and SDBS are not magnetic materials. The magnetic materials are only the CFOs and chromium. The magnetic substances in the whole sample are in the ratio of 1:18.

The magnetic properties of CNT-CFO and CNT-CFO-K₂Cr₂O₇ were studied using VSM at real-time (RT) and compared for the changes in magnetic properties. Three VSM measurements were carried out; the first was for pristine CNT-CFO; the second was after adding 90 μl of DI water and dried at room temperature. The third measurement was after adding 90 μl of K₂Cr₂O₇ in DI water, which is 98 μg of chromium, and drying at room temperature. The magnetic properties were measured at a maximum applied field of 15 kOe.

The data was analyzed for middle and front non-spin flip and spin-flip to investigate the magnetic scattering and structural scattering up and down to the direction of the neutron spin concerning the externally applied magnetic field. Results indicated that the magnetic signal was separated from the structural scattering for samples containing chromium. The non-spin-flip sum (↓↓+↑↑) data is dominated by structural scattering. The non-spin-flip difference (↓↓−↑↑) is a measure of magnetism parallel to the applied field modulated by the structure factor of whatever the magnetic scattering. The spin-flip phase of the ↓↓ data went to zero if there is no net magnetism along the applied field direction. Finally, the spin-flip sum (↑↓+↑↓) gives a measure of the magnetic-only component of moments that don't follow the applied field. The sample, which contains chromium, shows enhanced magnetism. For the CNT-CFO, however, there's a significant non-spin-flip difference even in the low field that increases further in a high field. In any field greater than 0.007 Tesla before the low-field measurements, the samples measured were different and not in some way repeated. In other words, they have very different transmission values. In all four cases, there's a strong, magnetic-only spin-flip signal that corresponds to the size of the nanoparticle. In conclusion, the most obvious difference between the samples seems to be that the CNT-CFO-Cr is harder to saturate than the CNT-CFO without chromium.

Magnetic measurements revealed significant remnant and saturation magnetization changes with the introduction of a very minute amount of chromium in CNT-CFO (introduction of one in thousand parts of chromium in CNT-CFO). The VSM analysis indicated that the composites' saturation magnetization increased by 66.4%, as shown in the hysteresis loop. The CNT-CFO responded to chromium significantly compared to the other common heavy metals. The small-angle neutron scattering (SANS) investigation indicated that the net moment of about 33.3% magnetic enhancement was recorded in agreement with the VSM measurements shown in FIG. 3. Therefore, a CNT-CFO nanocomposite can specifically detect the metal chromium and be used as a potential magnetic nanosensor for hexavalent chromium. The coercivity of the composite CNT-CFO increased from 1.01 to 200 Oesterds (Oe) when infiltrated with a smaller amount of chromium, as shown in the inset of FIG. 3.

Comparing the hysteresis (B-H) loop of pristine CNT/CFO (red) and CNT/CFO/K₂Cr₂O₇ (black) (FIG. 3), the B-H curve of the composite contaminated with chromium have high saturation magnetization and remanence magnetization.

The same method was used for other heavy metals to demonstrate the sensitivity and specificity of CNT-CFO for magnetic properties.

Accordingly, there is provided according to an embodiment of the invention, a sensor for the detection of trace amounts of heavy metal in a liquid comprising heavy-metal free magnetic nanoparticle composites and a carrier, said magnetic nanoparticle comprising carbon nanotubes intercalated with CoFe₂O₄. Any one or more heavy metals may be detected, but the heavy metal detected is preferably chromium, arsenic, cadmium, mercury and lead, and most preferably Cr-IV. The sensor is capable of detecting presence of heavy metals liquids in amounts of about 1 ppm and greater.

The selected carrier may be any type of container, filter, or flat substrate. In the case of a container, the carrier may be, by way of non-limiting example, a test tube, petri dish, beaker or graduated cylinder. In the case of a filter, the carrier may be any kind of sieve, strainer, mesh or screen device that permits the flow of liquid over the magnetic nanoparticle composite. In the case of a substrate, the carrier may be any kind of substrate, for example a flat substrate to which the magnetic nanoparticle is attached, fixed, or bound, and which allows the magnetic nanoparticle to be easily exposed to and removed from a liquid sample. Suitable examples of substrates include mica, soda-lime glass (SiO₂), sapphire (Al₂O₃), and mylar.

According to further embodiments of the invention, there is also provided a method for removing one or more heavy metals from a liquid comprising contacting the liquid with magnetic nanoparticle composites comprising carbon nanotubes intercalated with CoFe₂O₄, allowing said magnetic nanoparticle composites to absorb said heavy metals, and separating said magnetic nanoparticle composites containing absorbed heavy metals from said liquid. According to one embodiment, the magnetic nanoparticle composite may be affixed to insoluble macro particulate materials through an immobilization process. The macro particles bearing the magnetic nanoparticle composite may be packed in a fixed column or fluid bed reactor system, and a heavy metal containing liquid continuously pumped through the system. Heavy metals are there by removed from the liquid and the effluent liquid may be free of, substantially free of, or reduced in concentration of heavy metals. The reactor system may then be treated to remove a highly concentrated heavy metal solution for further processing, thereby regenerating the bound magnetic nanoparticle composite for reuse. As above, any heavy metal may be detected according to the various embodiment of the invention, but preferred heavy metals for detection are chromium, arsenic, cadmium, mercury and lead. According to a most preferred embodiment, the removed heavy metal is Cr-IV. Using this method, a heavy metal can be removed from a liquid, where the heavy metal is present in amounts as low as 1 ppm.

According to yet another embodiment of the invention, there is provided a method for detecting trace amounts of heavy metal in a liquid comprising contacting a sample of said liquid with a magnetic nanoparticle composite comprising carbon nanotubes intercalated with CoFe₂O₄ and measuring the neutron scattering, X-ray diffraction and/or magnetic properties of the magnetic nanoparticle composite, where a statistical difference in the neutron scattering, X-ray diffraction, X-ray absorption, or magnetic properties of the magnetic nanoparticle composite before and after contact between the sample of liquid and the magnetic nanoparticle composite indicates the presence of a heavy metal in the liquid. As above, any heavy metal may be detected, but preferred heavy metals for detection are chromium, arsenic, cadmium, mercury and lead. According to a most preferred embodiment, the detected heavy metal is Cr-IV. Using this method, heavy metals can be detected in amounts as low as 1 ppm. The neutron scattering measurements may be preferably carried out using small-angle neutron scattering (SANS). The magnetic properties may be preferably measured using vibrating multiple magnetometry (VSM). The X-ray absorption properties may be preferably measured by Extended X-Ray Absorption Fine Structure (EXAFS).

### BRIEF DESCRIPTION OF THE DRAWINGS

The foregoing summary, as well as the following detailed description of the preferred invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1A shows polarization analyzed small-angle neutron scattering on the vSANS instrument involving the neutron spin polarization components of a super-mirror, radiofrequency spin flipper, and a ³He spin analyzer. The neutron spins follow the applied magnetic field as shown unless they encounter abrupt magnetic changes such as within the sample.

FIG. 1B shows EXAFS instrumental anatomy.

FIG. 2 shows an X-ray diffraction (XRD) of CFO (black), CFO/CNTs (red), and CFO/CNT-Cr (blue). CFO peaks are in red, and Cr peaks are in blue diamond scatter.

FIG. 3 is a hysteresis loop of CFO/CNT (red) and CFO/CNT-K₂Cr₂O₇ (black), magnetization M in emu/g of CFO, and applied field in Oesterd (Oe). The inset on the top left compares the coercive field of CFO/CNT (red) and CFO/CNT-K₂Cr₂O₇ (black). The inset on the lower right is the hysteresis loop at a lower applied magnetic field of the VSM of CFO/CNT (red) and CFO/CNT-K₂Cr₂O₇ (black).

FIG. 4A shows polarization-analyzed SANS of CFO/CNTs and (b) CFO/CNT-Cr. The blue and gray curves are dominated by structural scattering and do not change significantly with an applied magnetic field. Red and purple curves are proportional to the magnetic scattering from moments aligned with the applied field at 0.007 T and 1.5 T, respectively. The gold curves arise from a scattering of magnetic moments not aligned with the magnetic field at 0.007 T. In contrast, the green curves show that almost no scattering is present from moments not aligned with the applied field at 1.5 T. Error bars represent one standard deviation.

FIG. 4B shows polarization-analyzed SANS of CFO/CNT-Cr. The blue and gray curves are dominated by structural scattering and do not change significantly with an applied magnetic field. Red and purple curves are proportional to the magnetic scattering from moments aligned with the applied field at 0.007 T and 1.5 T, respectively. The gold curves arise from a scattering of magnetic moments not aligned with the magnetic field at 0.007 T. In contrast, the green curves show that almost no scattering is present from moments not aligned with the applied field at 1.5 T. Error bars represent one standard deviation.

FIG. 5A shows magnetic-only SANS of CFO/CNTs.

FIG. 5B shows magnetic-only SANS of CFO/CNT-Cr. The primary difference between the sample of CFO/CNT (FIG. 5A) and the sample of CFO/CNT-Cr (FIG. 5B) is that a measurable scattering curve exists for the CNT-CFO-Cr sample at 0.007 T [red curve in FIG. 5B], which is absent in the CNT-CFO sample (FIG. 5A).

FIG. 6 shows EXAFS measurements showing the difference in the Fourier transform of CFO with Cr (blue) and Cr (red).

FIG. 7A shows EXAFS measurements showing the wave vector comparison of the pristine CFO/CNTs for Fe and CFO/CNTs with Cr for the energy of Fe.

FIG. 7B shows EXAFS measurements showing radial distance comparison of the pristine CFO/CNTs for Fe and CFO/CNTs with Cr for the energy of Fe.

### DETAILED DESCRIPTION OF THE INVENTION

CFO/CNT nanocomposite samples were prepared from a two-to-one ratio of CFO nanoparticles of the mean diameter of 42 nm and 10 μm-50 μm length CNTs as follows: CNTs (Cheap Tubes Inc.), SDBS surfactant, and CFO nanoparticles (Sigma-Aldrich) were used as received. Ultrasonication was performed using a Branson 450 Digital Sonifier with a ½″ disrupter horn. Sample preparation was initiated by dispersing 3.75 g of SDBS into DI water of resistivity measuring 18 Me-cm and sonicated for 20 minutes until a clear solution was achieved. Then, 0.5 g CNTs were added into this solution and sonicated for additional 20 minutes. Lastly, 0.25 g of CFO was added to this solution and sonicated further for 40 minutes. The solution was filtered and dried inside a vacuum oven at 80° C. for 10 hours at a pressure of 15 inches of mercury.

Then, 0.2 g of K₂Cr₂O₇ (Sigma-Aldrich), which contains 0.077 μg of chromium, was mixed in 65 ml of deionized water by magnetic stirring for 1 h, and 90 μl of the solution was transferred to the CFO/CNT composite to disperse over the surface of CFO/CNT nanoparticles. CFO/CNT and CFO/CNT-Cr (resulting in a 0.02 ratio of Cr/CoFe₂O₄ and 0.31 ratio of Cr/Co) samples were prepared for Small-Angle Neutron Scattering (SANS) and EXAFS characterization.

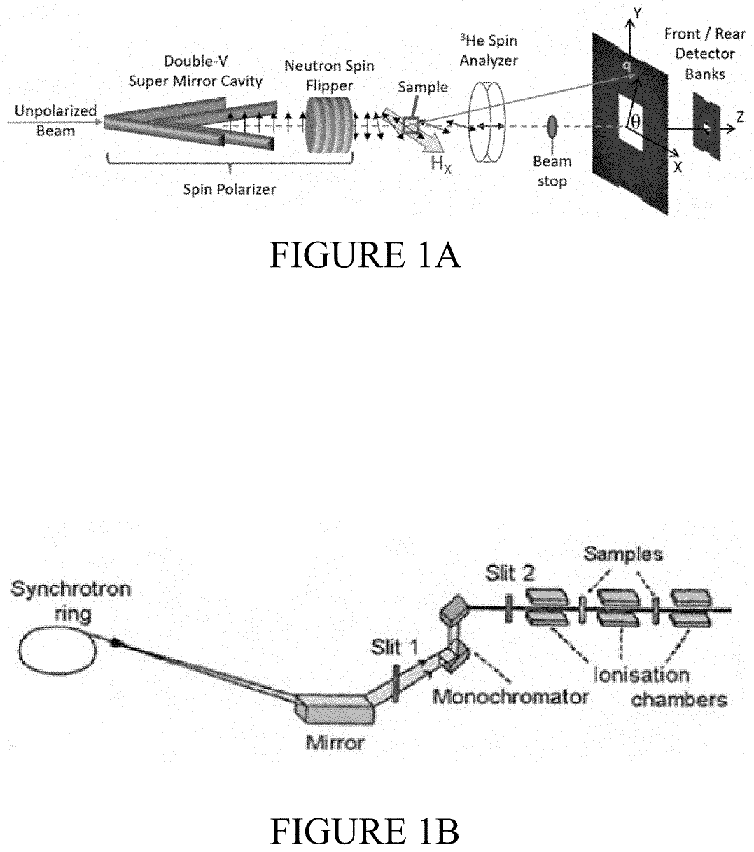

SANS samples were powder-packed into thin-walled Al sample cans and measured on the vSANS beamline at the NIST Center for Neutron Research at a wavelength of 0.55 nm and a full-width at the half-maximum wavelength spread of 12%. Data were simultaneously collected in two-dimensional detector banks located at 4.9 m and 17.4 m from the sample position, covering a reciprocal spae range of 0.003 Å⁻¹ to 0.12 Å⁻¹ (probing structures on the order of 5 nm-200 nm). The samples were placed in between the poles of a horizontal electromagnet yielding spatially uniform fields between 0.007 T (remanence) to 1.5 T, as shown in FIG. 1A.

Neutron scattering probes the ensemble averages of both structural (i.e., nuclear) and magnetic morphologies. However, in cases where the structural scattering dominates over the magnetic scattering, neutron spin polarization analysis can be used to effectively separate these two components and highlight the directional dependence of the magnetic scattering. Here, ↑ and ↓ represent neutrons whose spins are parallel and anti-parallel, respectively, to an applied external magnetic field. Neutron spin polarization was selected prior to the interaction with the sample via an in-beam FeSi super-mirror polarizer cavity and a radio frequency spin flipper, while the relative amounts of spin polarization after scattering from the sample were measured with a ³He neutron spin filter combined with an in situ NMR flipper. The efficiency of each polarizing element, though high, is fully corrected. This results in a total of four spin cross sections: ↑↑, [symbol] , ↓↓, and [symbol] , where the arrows refer to neutron spins before and after the sample, respectively. In short, ↑↑+↓↓ taken along the vertical direction (⊥H and ∥Y in FIG. 1A) measure structural scattering plus scattering from moments aligned parallel to H, while [symbol] + [symbol] taken along the vertical direction measures only scattering from magnetic moments perpendicular to H. The latter is multiplied by a factor of two to account for the fact that this procedure measures only half the moments not aligned along H (i.e., moments ∥Z, but not moments ∥Y).

Additionally, ↓↓−↑↑ also taken along the vertical direction [⊥H and ∥Y in FIG. 1A] is a measure of structural-magnetic cross-term involving moments aligned ∥H. In practice, sector cuts of ±15° were taken about the vertical (Y) direction, where the component of M∥H is proportional to sin²(θ) and θ is the angle from the positive x-axis shown in FIG. 1A.

EXAFS is an advanced and widely used method for studying atoms and their local environments. EXAFS uses the x-ray photoelectric effect and the wave nature of the electron to determine local structures around the atom in solid and nanomaterials. EXAFS has become more applicable to investigate the electrochemical and magnetic nature of magnetic nanoparticles. The EXAFS area from the entire range is characterized by a function χ, defined in terms of the absorption coefficient, as shown in the following equation:

$$χ ( E ) = \frac{μ ( E ) - μ 0 ( E )}{μ o ( E )} , \qquad ( 1 )$$

where μ(E) is a function of energy or excess energy, and μ₀(E) is the initial x-ray absorption energy at the edge. EXAFS is a technique used to measure the molecular parameters of materials and to study the local structures and movements of atoms during chemical reactions. Here, a high penetration depth by fine-tuning the EXAFS energy range was achieved.

The QAS 7BM beamline at the National Synchrotron Light Source II (NSLS-II) of Brookhaven National Laboratory (BNL) as shown in the schematic diagram in FIG. 1B was used to investigate the magnetic characteristics of the CFO/CNTs infiltrated with Cr-VI. Our samples were prepared based on a requirement set by the BNL for EXAFS measurements.

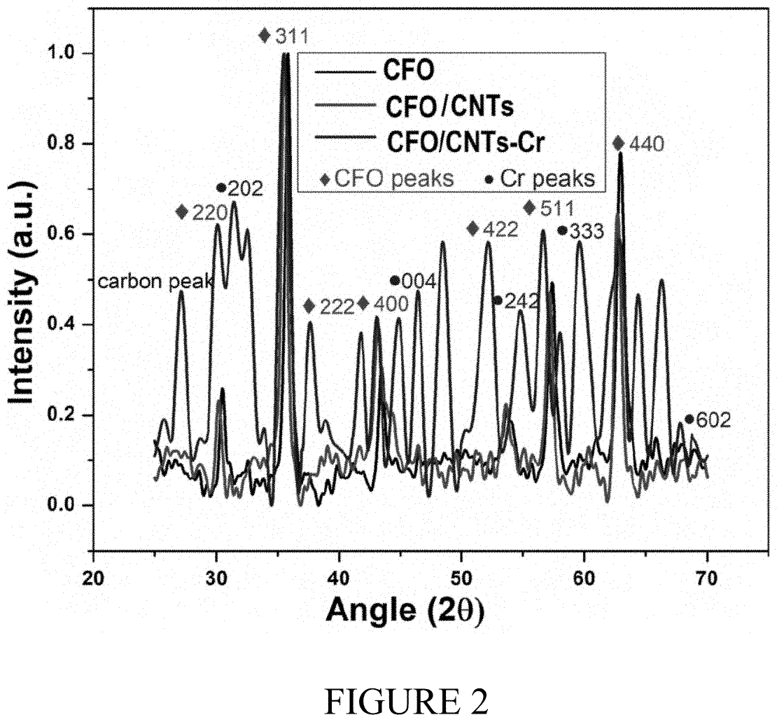

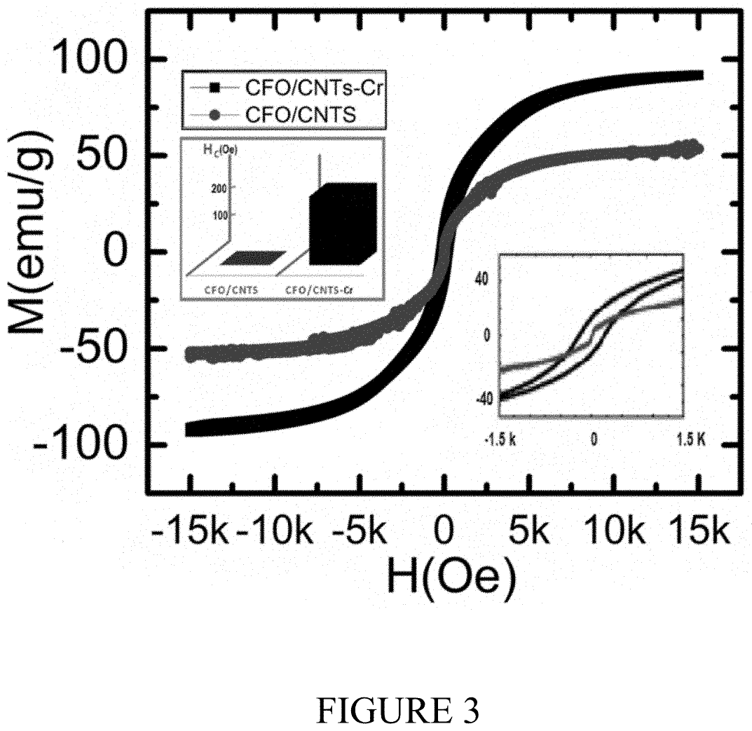

Neutron scattering of magnetic materials, such as CFO, provides essential information on the magnetic and nuclear cross sections. XRD of CFO, CFO/CNTs, and CFO/CNTs-Cr is shown in FIG. 2 with the corresponding CFO and Cr peaks. The magnetic characteristics, especially saturation magnetization of CFO/CNTs infiltrated by a minute amount of Cr-VI, exhibit 71% enhancement compared to the pristine CFO/CNT nanocomposites as shown in FIG. 3.

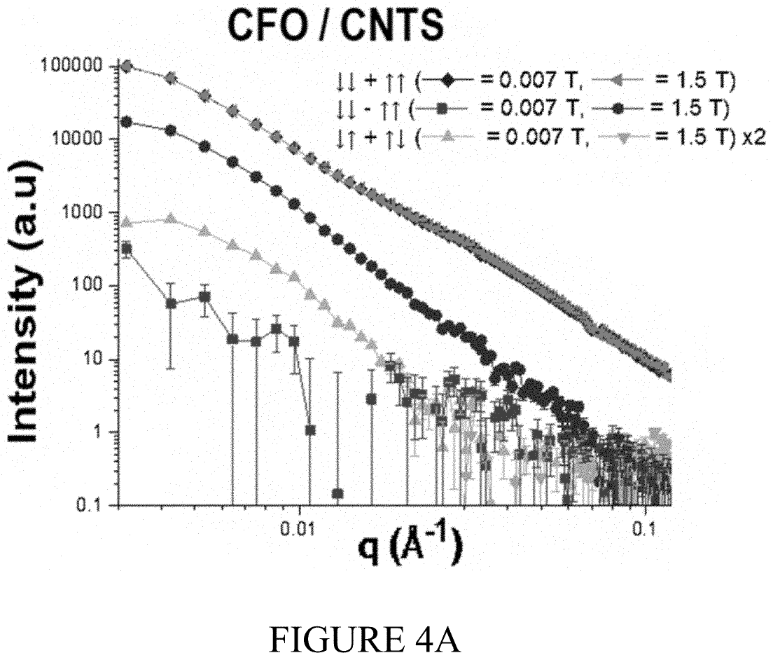

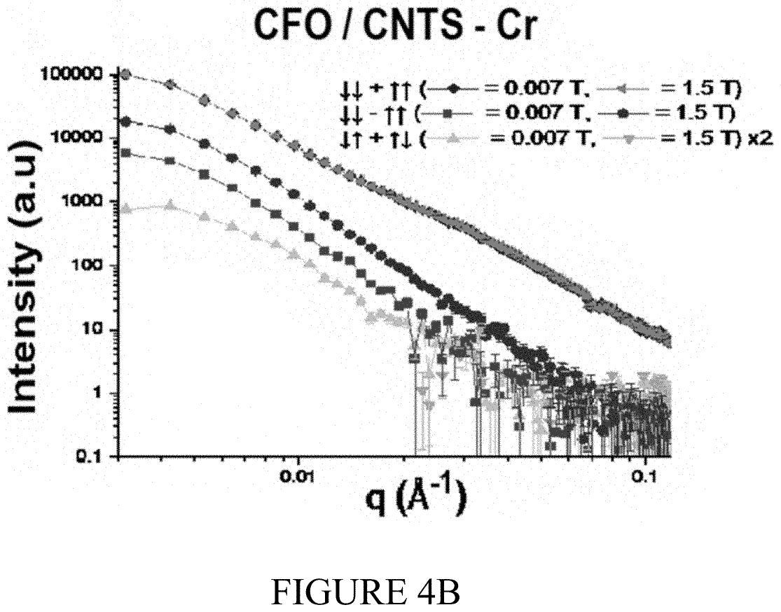

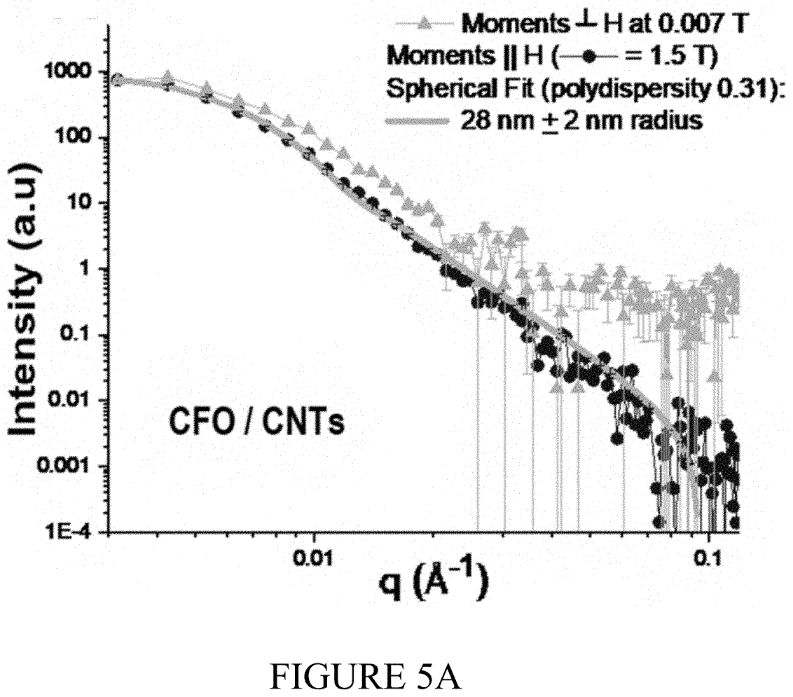

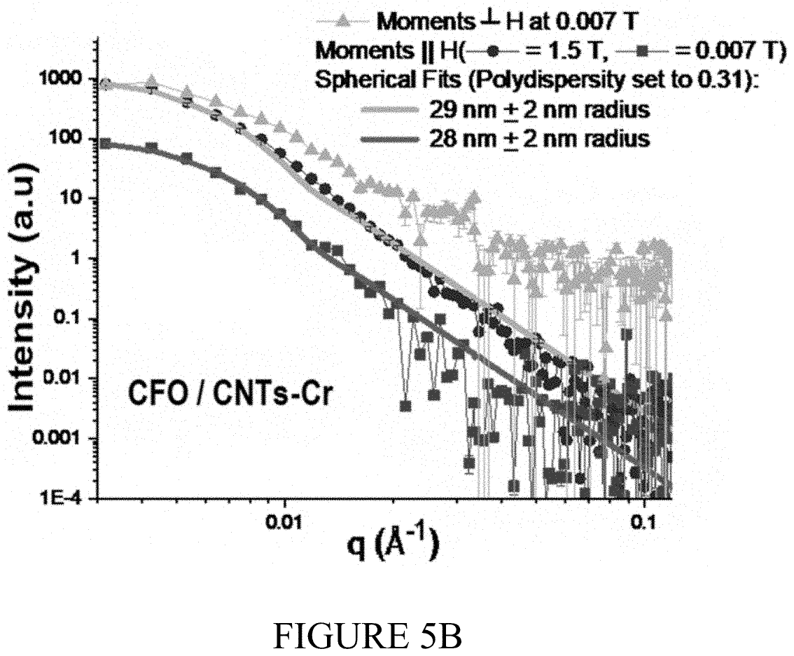

The polarized SANS data from CFO/CNTs and CFO/CNT-Cr samples are shown in FIGS. 4A and 4B. The precise scaling between the two samples is not known due to variations in powder packing. Thus, a uniform correction was applied to each sample to bring the structural scattering at 0.007 T at the lowest Q-measurement of 0.003 Å−1 to even 100,000 counts (for the ease of direct comparison). In both the CFO/CNTs and CFO/CNT-Cr samples, we see that there is no significant difference between ↓↓+↑↑ scattering at 0.007 T and 1.5 T, indicating that the sample scattering is dominated by structural contributions rather than magnetic scattering contributions (blue and gray curves of FIGS. 4A and 4B). It is also evident in both samples that the magnetic-only spin-flip scattering ( [symbol] + [symbol] ), which arises from the component of magnetic moments not aligned with the applied external field, is significant at 0.007 T (though a factor of about 100 lower than the structural scattering), but not at 1.5 T (data points in yellow and green in FIGS. 4A and 4B). This implies that at 1.5 T, the magnetic moments are fully aligned with the applied field. The (↓↓−↑↑) scattering is similar for the two samples at 1.5 T, yet differs markedly at 0.007 T. The fact that this signal, proportional to moments ∥H, is negligible in the CFO/CNTs at 0.007 T means that almost no net moment ∥H persists at remanence. Yet, its presence in the CFO/CNT-Cr sample indicates that a net magnetization persists at remanence. This is in agreement with the magnetometry results in FIG. 3. Moreover, in the CFO/CNT-Cr, the (↓↓−↑↑) signal is 3.1±0.1 times larger at low Q (up to 0.01 Å⁻¹) at 1.5 T than at remanence, which is also in general agreement with magnetometry. However, the ratio of (↓↓−↑↑)/(↓↓+↑↑) at 1.5 T of FIGS. 5A and 5B is proportional to the magnetism ∥H/structural scattering, and it is very similar in the samples with peak ratios of 0.20 at 0.005 Å⁻¹. This suggests that the saturation magnetization is almost the same for the CFO/CNTs and CFO/CNT-Cr samples within the reciprocal space probed by SANS. Instead, the large saturation magnetization enhancement measured by VSM with the addition of Cr-IV might be correlated with very large magnetic clusters.

The magnetic scattering can be further refined into components arising from magnetism ⊥H and magnetism ∥H, see FIGS. 5A and 5B. Assuming that the magnetism ∥H is correlated with the structural scattering, the magnetic scattering ∥H can be estimated as ¼ (↓↓−↑↑)2/(↓↓+↑↑), as shown in FIGS. 5A and 5B (blue and red curves). Again, the magnetic scattering at 1.5 T is similar in magnitude and shape for both the CFO/CNTs and CFO/CNT-Cr samples. Using SasView, this can be fit to magnetic nanoparticle spheres of radius (28±2) nm and (29±2) nm, respectively, with a 0.31 full-width half-maximum polydispersity. The residual magnetic moments at 0.007 T for the CFO/CNT-Cr sample also fit a similar model of (28±2) nm magnetic spheres. Here, the difference between the magnetic scattering at 0.007 T and 1.5 T is a function of the magnetic moments squared. While the magnetic model predicts magnetic particles slightly larger than the expected 42 nm in diameter, we note that the modeling is highly dependent upon the polydispersity of the nanoparticles such that more polydispersity would yield a lower mean diameter. The models do reveal a consistent magnetic size for the moments aligned ∥H across the samples and at different applied magnetic fields. Finally, the moments perpendicular to H (gold curves of FIGS. 5A and 5B) have a much higher spin-flip background, likely due to the incoherent scattering associated with hydrogen (which is removed from the processing of the magnetism ∥H scattering during the ↓↓−↑↑ subtraction step). However, the scattering from these moments perpendicular to H at 0.007 T is similar in magnitude to that from the moments ∥H at 1.5 T, showing rough conservation of moments and a tendency for the moments to stay aligned within the individual nanoparticles even as remanence.

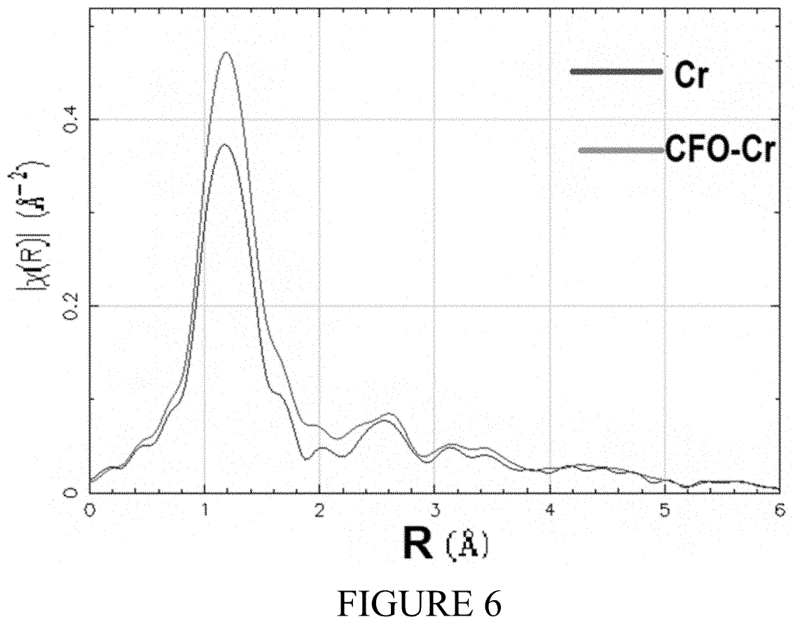

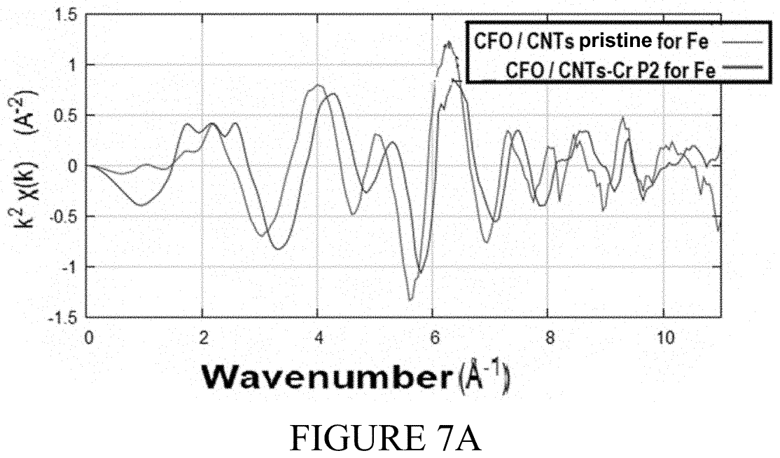

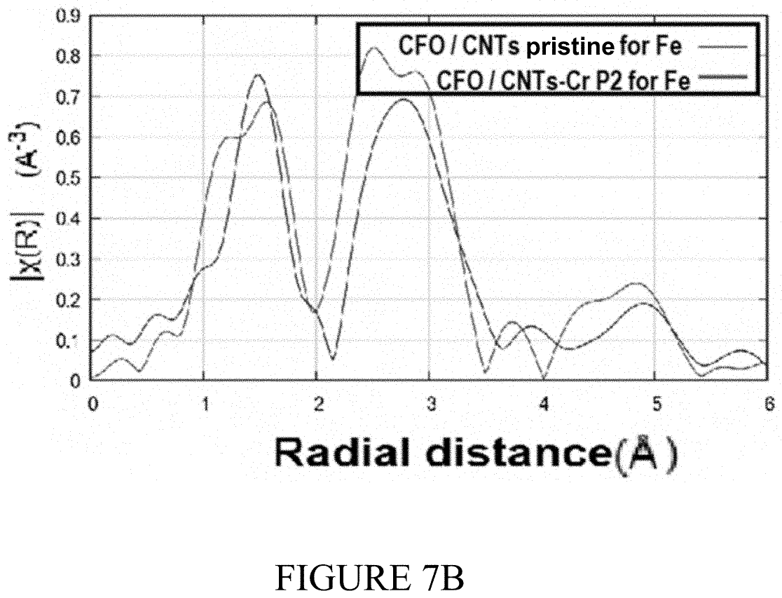

The EXAFS characterization of CFO/CNTs infiltrated with Cr-VI turns out to be radically different from the one observed for pristine CFO/CNTs. The fingerprinting analysis of the data is shown in FIGS. 6, 7A and 7B. The data range was from −200 eV to 525 eV, relative to the K-edge of iron, and the k range was from 3 to 11 using the Hanning window, and the k2 weighting was used. The difference can be explained either in terms of distinct intercalation sites between both samples or in terms of different interactions between O, Fe, Co, and Cr.

As a general principle, the x-rays of specific energy are absorbed, removing a core electron of the K-shell. In the test sample, the core electron was from the S-shell of iron. The x-ray energy to knock out this electron is about 7112 eV. As the x-ray energy keeps increasing, this electron wave keeps taking the excess energy and interacts with surrounding atoms and scatters back to the absorbing atom. The constructive and destructive interference at the absorber gives rise to the EXAFS pattern. Mathematical massaging of this pattern infers information about the atomic distances and coordination numbers.

The CFO has a spinel crystal structure. The O and Fe atoms are at tetrahedral and octahedral sites. The Fourier transform of the CFO/CNT pristine original for Fe indicates that about 40% occupy the tetrahedral site and 60% occupy the octahedral site. The structure of CFO, using EXAFS, has been reported for two Fe—O, Fe—Fe, Fe—Co, and Co—O distances with their coordination numbers for this spinel crystal, calcined at 800° C. The reported distances are Fe—O—1.88 Å, Fe—O—1.99 Å, Fe—Fe—2.96 Å, and Fe—Co—3.47 Å, and their observed coordination numbers are 1.87, 0.86, 3.15, and 2.11, respectively.

Fundamental differences were observed in the Fe data of the two datasets, CFO/CNTs not infiltrated by Cr-VI, and pristine CFO/CNTs. The radial distribution plot of CFO/CNTs in FIG. 7B, without phase correction, shows the first peak at 1.49 Å; this is the peak corresponding to the Fe—O distance in the sample as deposited on carbon nanotubes. The second peak at 2.78 Å is likely the Fe—Fe distance. These peaks split when Cr-VI is added. The first peak splits into two, at 1.22 Å and 1.56 Å. Either the Fe—O distance has been reduced, and an O is at 1.22 Å and Cr at 1.56 Å, or it has been expanded, and a Cr atom is at 1.22 Å and O at 1.56 Å. In the CFO/CNT sample, the second peak was at 2.78 Å. With the addition of Cr-VI, the distance splits to 2.50 Å and 2.84 Å, which is one lower and one higher distance from the original second shell of iron in the CFO/CNT sample. Either the lower one is a shrunk Fe atom and the higher one a Cr atom, or it may be vice versa. The peak height differences of the two major peaks of the radial distribution, when adding K₂Cr₂O₇, also indicate a redistribution of the octahedral and tetrahedral sites—apparently about 90% tetrahedral and 10% octahedral.

Similar results are found with arsenic, cadmium, mercury and lead.

The infiltration of Cr-VI into cobalt ferrite magnetic nanoparticles intercalated on carbon nanotubes (CFO/CNTs) reveal useful magnetic differences at both saturation and remanence, demonstrating that CFO/CNTS composites can be harnessed to detect environmental contamination by Cr and other heavy metals. EXAFS show structural changes between CFO/CNTs with and without Cr-IV infiltration, indicating that the Cr has been incorporated into the CFO structure. At magnetic saturation (1.5 T), VSM showed a significant 71% magnetic enhancement in the CFO/CNT composite containing Cr-IV, while the SANS showed that this magnetic difference was not correlated with the local magnetic enhancement of the cobalt ferrite nanoparticles. Thus, the VSM may be sensitive to a magnetic enhancement correlated with much larger structures. At remanence (up to 0.007 T), both the VSM and SANS revealed that the CFO/CNT-Cr nanoparticles were hysteretic with a residual magnetization about ⅓ that of saturation, while the CFO/CNT composites were not hysteretic and did not contain a net remanent magnetization. Thus, the CFO/CNT architecture disclosed herein offers two ways to detect environmental heavy metal contamination: through an increase in the long-range net magnetization at saturation or by imparting a residual magnetization within the CFO nanoparticles after exposure to a magnetic field.

It will be appreciated by those skilled in the art that changes could be made to the preferred embodiments described above without departing from the inventive concept thereof. It is understood, therefore, that this invention is not limited to the particular embodiments disclosed, but it is intended to cover modifications within the spirit and scope of the present invention as outlined in the present disclosure and defined according to the broadest reasonable reading of the claims that follow, read in light of the present specification.

1. A sensor for the detection of trace amounts of heavy metal in a liquid comprising heavy-metal free magnetic nanoparticle composites and a carrier, said magnetic nanoparticle comprising carbon nanotubes intercalated with CoFe₂O₄.

2. A sensor according to claim 1, wherein said heavy metal is selected from the group consisting of chromium, arsenic, cadmium, mercury and lead.

3. A sensor according to claim 1, wherein the heavy metal is Cr-IV.

4. A sensor according to claim 1, capable of detecting presence of heavy metals liquids in amounts as low as about 1 ppm.

5. A sensor according to claim 1, wherein said carrier is selected from the group consisting of containers, filters, or flat substrates.

6. A sensor according to claim 5, wherein said carrier is a container, and said container is a test tube, petri dish, beaker or graduated cylinder.

7. A sensor according to claim 5, wherein said carrier is a filter.

8. A sensor according to claim 5, wherein said carrier is a substrate.

9. A sensor according to claim 5, wherein said substrate is selected from the group consisting of mica, soda-lime glass (SiO₂), sapphire (Al₂O₃), and mylar

10. A method for removing heavy metal from a liquid comprising contacting said liquid with magnetic nanoparticle composites comprising carbon nanotubes intercalated with CoFe₂O₄, allowing said magnetic nanoparticle composites to absorb said heavy metals, and separating said magnetic nanoparticle composites containing absorbed heavy metals from said liquid.

11. A method according to claim 10, wherein said heavy metal is selected from the group consisting of chromium, arsenic, cadmium, mercury and lead.

12. A method according to claim 10, wherein the heavy metal is Cr-IV.

13. A method according to claim 10, where the heavy metal is present in in amounts as low as about 1 ppm.

14. A method for detecting trace amounts of heavy metal in a liquid comprising contacting a sample of said liquid with a magnetic nanoparticle composite comprising carbon nanotubes intercalated with CoFe₂O₄ and measuring the neutron scattering, X-ray diffraction and/or magnetic properties of the magnetic nanoparticle composite, where a statistical difference in the neutron scattering, X-ray diffraction, X-ray absorption, or magnetic properties of the magnetic nanoparticle composite before and after contact between said sample of said liquid and said magnetic nanoparticle composite indicates the presence of a heavy metal in said liquid.

15. A method according to claim 14, wherein said heavy metal is selected from the group consisting of chromium, arsenic, cadmium, mercury and lead.

16. A method according to claim 14, wherein the heavy metal is Cr-IV.

17. A method according to claim 14, capable of detecting presence of heavy metal in liquids where the amount of heavy metal is present in amounts as low as about 1 ppm.

18. A method according to claim 14, wherein neutron scattering measurement is carried out using small-angle neutron scattering (SANS).

19. A method according to claim 14, wherein magnetic properties are measured using vibrating multiple magnetometry (VSM).

20. A method according to claim 14, wherein X-ray absorption properties are measured by Extended X-Ray Absorption Fine Structure (EXAFS) characterization.
